(19) United States
(12) Patent Application Publication
(10) Pub. No.: US 2007/0254319 A1
(43) Pub. Date: Nov. 1, 2007

(54) IDENTIFICATION OF A CONSTITUTIVELY RESISTANT CANCER STEM CELL

(75) Inventors: Vera Svobodova Donnenberg, Pittsburgh, PA (US); Albert David Donnenberg, Pittsburgh, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: University of Pittsburgh - Of the Commonwealth System of Higher Education, Pittsburgh, PA (US)

(21) Appl. No.: 11/733,114

(22) Filed: Apr. 9, 2007

Related U.S. Application Data
(60) Provisional application No. 60/790,324, filed on Apr. 7, 2006.
(60) Provisional application No. 60/801,293, filed on May 18, 2006.

(51) Int. Cl. G01N 33/574; C12N 5/08

(52) U.S. Cl. 435/7.23; 435/40.5; 435/366

(57) ABSTRACT

In one embodiment, the invention provides a method of identifying and isolating a cancer MDR stem cell. In another embodiment, the invention provides an isolated cancer MDR stem cell and a population of such cells. In yet another embodiment, the invention provides a method of screening a test compound for its ability to kill or impede proliferation of MDR cancer stem cells.

### CROSS-REFERENCE TO RELATED APPLIATIONS

This application claims priority to U.S. Provisional Patent Application 60/790,324, filed Apr. 7, 2006. This application also claims priority to U.S. Provisional Patent Application 60/801,293, filed May 18, 2006. The contents of these priority applications are incorporated herein in their entirety.

### STATEMENT CONCERNING FEDERALLY SPONSORED RESEARCH

Research leading to this invention was funded, in part, through grants from the United States Department of Defense under award numbers BC044784, and BC032981. The Government of the United States of America may have certain rights in this invention.

### BACKGROUND OF THE INVENTION

Multiple drug resistance (MDR) is recognized as an important barrier to cancer chemotherapy. Currently, MDR is thought to result from drug selection and gene duplication or rearrangement. Thus, while existing technology permits the identification of drug resistance in bulk tumor tissue, it does not afford the ability to identify MDR cancer cells prior to treatment.

However, the existence of MDR cancer stem cells has been suggested (See Donnenberg V S, and Donnenberg A D. Multiple drug resistance in cancer revisited: the cancer stem cell hypothesis. J Clin Pharmacol. 2005 August;45(8):872-7). While not intending to be bound by theory, it is suggested that the MDR cancer stem cell is a resting cell with drug resistance that is not dependent on therapy-induced gene duplication or translocation. This cell, thus, has the capacity to generate mitotically active, drug-sensitive tumorigenic daughter cells as well as drug insensitive cancer stem cells through asymmetric division.

While the existence of MDR cancer stem cells has been suggested, existing technology does not afford a method of identifying such cells. Accordingly, improved diagnostic methods are needed to identify MDR cancer stem cells.

### BRIEF SUMMARY OF THE INVENTION

In one embodiment, the invention provides a method of identifying and isolating a cancer MDR stem cell. In another embodiment, the invention provides an isolated cancer MDR stem cell and a population of such cells. In yet another embodiment, the invention provides a method of screening a test compound for its ability to kill or impede proliferation of MDR cancer stem cells. These and other advantages of the invention, as well as additional inventive features, will be apparent from the accompanying figures and the description of the invention provided herein.

### DESCRIPTION OF THE FIGURES

FIG. 1 depicts the process for tissue digestion and staining of cells in accordance with the inventive method.

FIG. 2 depicts data demonstrating stem cell marker expression on ABCG2+ cells from normal lung and therapy naïve epithelial cancers. The top row shows the gating strategy used for all analyses: Singlet cells were selected in a plot of forward scatter pulse height by forward scatter pulse width; apoptotic cells and debris were removed on a plot of forward scatter by log side scatter; and CD45 negative (non-hematopoietic) cells were selected on a plot of CD45 by log side scatter. This compound gate was applied to histograms of intracellular cytokeratin by ABCG2. The second row shows representative plots for lung tissue (grossly normal tissue resected from the lung of a patient with NSC lung cancer), a pleural effusion (untreated metastatic NSC lung cancer), a malignant ascites (untreated ovarian adenocarcinoma), and a lung tumor (NSC lung Ca). The numbers in the upper right hand quadrants represent cytokeratin+ ABCG2+ cells, expressed as a percent of CD45− live singlet cells (G2+ Cytok+ frequency). The bar graphs show the frequency, and proportion of cells expressing resting morphology (low forward and side light scatter), CD44, CD117, CD90, and CD133, by tissue origin. Bars indicate 1 SE above the mean values.

FIG. 3 is a flowchart demonstrating the manner of classification of stem, progenitor and mature progeny from digested tissues and tumors. This strategy has been used for both analysis and sorting. The 4-way sort option of the MoFlo sorter allows all 4 classes (red) to be collected simultaneously. Outcome variables, such as MDR expression and activity, and epithelial and neuroendocrine differentiation markers are measured on each class. It should be noted that the unclassified population accounts for <5% of live CD45− cells.

FIG. 4 depicts data demonstrating the detection of CD45− cytokeratin+ cells expressing stem cell-associated markers in normal and neoplastic lung parenchyma and in two malignant effusions.

FIG. 5 depicts staining data demonstrating that CD45− CD90+ cells isolated from primary tumors have small resting morphology. A freshly resected untreated NSC lung Ca was digested with collagenase and stained with CD45, CD90 (green) and the nuclear stain Draq5 (red). Images were collected with the Amnis ImageStream imaging flow cytometer. The top panel (A) shows images of nonhematopoietic (CD45−), CD90+ cells. The bottom panel (B) shows photomicrographs from CD45− CD90− cells.

FIG. 6 depicts data demonstrating constitutive MDR transport in small resting CD45− CD117+ tumor cells from untreated ovarian malignant ascites. Live (PI negative), singlet, CD45− cells were gated on CD117. R123dim CD117+ cells were color evented red; R123bright CD117+ cells were color evented blue. The light scatter profile (bottom right) clearly shows that constitutive MDR activity as evidenced by R123 efflux, is uniquely localized to the small resting phenotype (red).

FIG. 7 depicts data demonstrating that sorted lung-tumor derived CD90+ stem cells are self-renewing and form embryoid bodies. CD45− primary NSC lung tumor cells (left panel, 10,000 cells/well), and CD45− CD90+ (100 cells/well) were cultured under ES conditions. Sorted CD45− bulk tumor rapidly formed clusters of proliferating cells that developed into structures resembling embryoid bodies surrounded by fibroblasts by day 14. The right panel shows that sorted CD45− CD90+ cells (100 cells/well) gave similar results. Cells in the right panel were passaged and expanded in culture conditions optimized for embryonic stem cells (ES). Flow cytometry at day 21 (lower panels) shows marked expansion of the stem cell compartment.

FIG. 8 depicts data demonstrating that human CD45− CD90+ ABGG2+ cancer stem cells isolated from a malignant effusion produce tumors in SCID/NOD mice. The animal shown was sacrificed at 8 months with tumors at 4/4 injection sites (2 shown with arrows). The enlarged area shows a highly vascularised subcutaneous tumor. This tumor was disaggregated stained and resorted for CD45− CD90+ ABCG2+ cells (box). These cells were injected into SCID/NOD mice. As of 4 months post-injection, 4 of 5 re-sorted mice have developed large tumors at the injection site.

FIG. 9 depicts data concerning the simultaneous detection of the ABC transporters ABCB1 and ABCG2. Flow cytometry was performed on the parental cell line K562 (bottom panels, human erythroleukemia) and the MDR1 transfectant K562-G185 (top panels). A gating strategy was used to analyze only singlet viable cells. Parental K562 cells were ABCB1 (MDR1) negative and expressed a very small subpopulation (0.08% of viable cells) of ABCG2+ cells having low side scatter. This population was not detectable by RT PCR, owing to its scarcity. In contrast, the transfectant line K562-G158, which is maintained in the presence of 100 ng/mL vincristine, is uniformly positive for ABCB1, and expresses an identical small subpopulation of ABCG2+ cells.

FIG. 10 depicts data concerning the simultaneous measurement of Hoechst 33342 (Ho33342) and R123 dye efflux in MDR1 transfected (G185) and parental K562 cells. Cells were stained with anti-CD117 and incubated for 90 min in the presence of Ho33342 (8 microM, ˜5 microg/mL) and R123 (0.13 microM) with and without inhibitors. PI was added immediately before acquisition on the MoFlo flow cytometer in order to eliminate dead cells. Events were gated to exclude cell clusters and dead cells. The side population (SP) phenotype (blue gate) and R123 excluding cells (single parameter histogram left of the dashed line) were measured on CD117− (top panels) and CD117+ (bottom panels). CD117+ cells comprised <1% of total cells.

CD117− Cells: The density plots (left panels) show that G185 MDR transfectants exhibited a large SP (52%), which was fully inhibited by cyclosporine (94% inhibition), partially inhibited by verapamil (69%) and not affected by the ABCG2-specific inhibitor fumitremorgin, or the MDR substrate drug vincristine. In contrast the native SP phenotype in parental K562 cells represented only 1.42% of cells and was equally inhibited by fumitremorgin, cyclosporine and verapamil. This indicates that the native SP phenotype in K562 cells is due to ABCG2 and not ABCB1. These findings are confirmed in the single parameter histograms of R123 fluorescence (right panels). Here, the SP cells (within the blue gate in the left plots) is color evented blue, whereas SP negative cells are shown in gray. The dotted trace shows total cells. These results show that virtually all SP cells also efflux R123. Inhibition R123 efflux mirrored that of Ho33342 efflux in G185 transfectants.

CD117+ Cells: The rare CD117+ population was enriched for SP cells in both transfectant and parental lines.

FIG. 11 depicts data demonstrating simultaneous detection of Hoechst 33342 and R123 transport in freshly isolated cells from a therapy naïve non-small cell lung tumor.

### DETAILED DESCRIPTION OF THE INVENTION

In one embodiment, the invention provides a method of identifying an MDR cancer stem cell. In accordance with the inventive method, first a tissue sample (e.g., biopsy) is obtained from a patient. For example, the sample can be a lymph node, or a portion of an organ (e.g., lung, breast, skin, etc.), which may be suspected of harboring cancerous or precancerous cells.

The tissue sample then is prepared for flow cytometry according to standard methods according to which single cells within the tissue sample can be stained for identification or purification. However, in accordance with the inventive method, the single cells within the tissue sample are stained with dye-conjugated antibodies (preferably monoclonal antibodies) for identification or purification by flow cytometry. Preferably, the antibodies target CD45, CD90, CD117, CD133, and a marker of multiple drug resistance (such as ABCG2 (mitoxantrone resistance, Breast Cancer Resistance Protein 1), ABCB1 (MDR1, P-glycoprotein), ABCC1 (Multiple Resistance Protein) and Lung Resistance Protein (LRP)).

CD45 is preferably employed to remove hematopoetic-derived cells. However, other hematopoetic-specific antibodies could be used as functional equivalents. For example, a cocktail of lineage-specific antibodies can be employed to identify lineage-negative non-hematopoetic cells. Such lineage “cocktails” are composed of antibodies directed against epitopes expressed by RBC (red blood cells), lymphocytes of T-, B- and NK-lineages (CD3, CD4, CD8, CD19, CD16, CD56), monocytes, macrophages and histiocytes (tissue macrophages), eosinophills and basophills, neutrophills and granulocytes, platelets, and their precursors (non-epithelial lineage commitment).

The antibodies for use in the inventive method can be prepared by standard methodology and/or are commercially available (e.g., through Beckman-Coulter, Becton-Dickinson, Invitrogen and Chemicon. Dyes are purchased from Sigma and Invitrogen.).

Following labeling with the antibodies, the stained cells preferably are cultured in the presence of fluorescent MDR substrates. For example, Rhodamine 123 and Hoechst 33342 are substrates for the MDR transporters ABCG2 and ABCB1, respectively, and preferably the cells are exposed to both of these substrates. Other fluorescent MDR substrates can be employed as well (e.g., MDR Assays Using Acetoxymethyl Esters, Vybrant Multidrug Resistance Assay Kit, Diagnostic Assay for Multidrug Resistance, MDR Assays Using Glutathione-Reactive Probes, MDR Assays Using Mitochondrial Probes (e.g. R123), MDR Assays Using Nucleic Acid Stains (e.g. Hoechst 33342), BODIPY FL Verapamil, BODIPY Dihydropyridines, BODIPY FL Paclitaxel, BODIPY FL Vinblastine, BODIPY Prazosin and BODIPY Forskolin, MDR Assays Using Ion Indicators, and the like). Typically, the cells are exposed to these fluorescent MDR substrates for 15-90 min, but any suitable time can be employed.

Following exposure to the fluorescent MDR substrates, a viability dye can be added to the cells, and preferably is added. Such dye can be, for example propidium iodide, DAPI, 7AAD, however, other suitable viability dyes can be used. Typically, viability dyes act very quickly. Thus, within a short period of time (at most, several minutes) following addition of the viability dye, the cells are subjected to flow cytometry. Preferably, the cells are subjected to the flow cytometry immediately after exposure to the viability dye.

Following the flow cytometry, MDR cancer stem cells can be identified as having a combination of some of the following factors: 1) Live (viability dye excluding); 2) Singlet (by forward light scatter pulse analysis; 3) Non-hematopoietic (CD45 negative); 4) CD90, CD133, or CD117 positive; 6) MDR expression and/or activity (for example ABCG1+, ABCB1+, ABCC1+ and/or LRP+); 7) Rhodamine 123 and/or Hoechst 33342 transport (and preferably excludes both dyes). The MDR cancer stem cells also can be CD44+.

Using flow cytometry, it also is possible to isolate the MDR cancer stem cell that has been identified using this method. Fluorescence activated cell sorting (FACS) alone using the markers described herein is sufficient to isolate MDR cancer stem cells from primary or metastatic tumor samples. Analytical flow cytometry can be used to detect the presence of these cells without isolation. For the detection and isolation cancer stem cells in the circulation, magnetic bead cancer stem cell enrichment and depletion of non-epithelial cells enhances sensitivity. Thus, the invention further provides a method of isolating an MDR cancer stem cell by employing the foregoing method and then culturing the MDR stem cell. Furthermore, the invention provides a substantially isolated MDR stem cell.

In accordance with the invention, MDR cancer stem cell is substantially isolated from non-MDR cancer stem cells of the same species of the MDR cancer stem cell. By “substantially” isolated, it is meant that the MDR cancer stem cell is the predominant cell type in the culture. Preferably, the inventive MDR cancer stem cell is free of contamination by other cell types. It should be noted, however, that in some embodiments, the inventive MDR cancer stem cell will be in the presence of substantial numbers of cells of a species other than the species of the MDR cancer stem cell. For example, the inventive MDR cancer stem cell can exist in vivo, such as a MDR cancer stem cell of human origin being placed into an animal model (e.g., a mouse host). In fact, it is preferred that the inventive MDR cancer stem cell tumorigenic at high frequency in such xenograft model systems. Most preferably, the MDR cancer stem cell is of human origin and is tumorigenic at a frequency of at least about 40 cells per injection site in SCID/NOD mice.

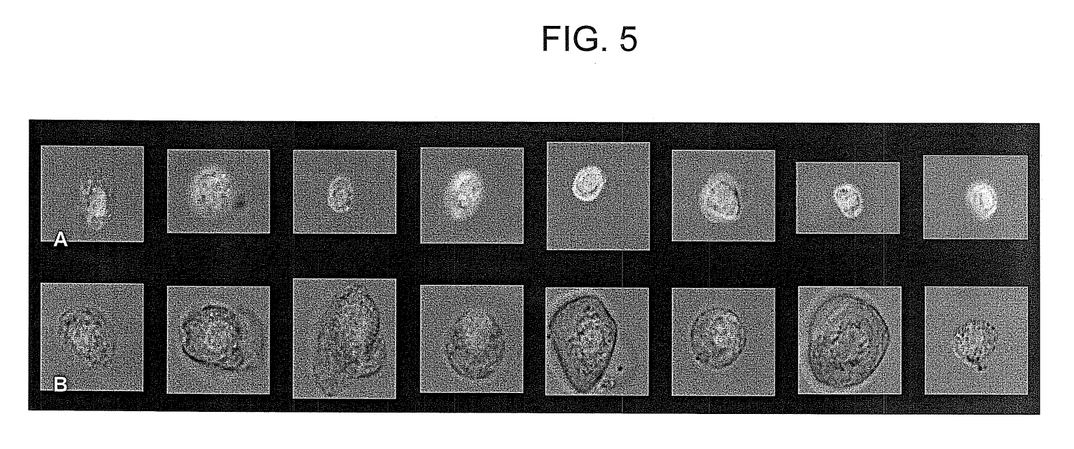

Another preferred property of the inventive MDR cancer stem cell is for it to bear stem-cell associated markers and/or progenitor-cell associated markers. The principle feature employed to distinguish resting stem cells from progenitor cells is morphology. In single cell suspension, resting stem cells are small round cells, with high nucleus/cytoplasm ratio. This corresponds to low forward and side light scatter by flow cytometry. Both stem and progenitor populations express CD117+, CD90+, and/or CD133+. They also have scant RNA, as can be measured by flow cytometry using acridine orange staining. Progenitor cells are large metabolically active cells with low nucleus/cytoplasm ratio and can be found in disaggregated normal and neoplastic lung at a low frequency (<0.1%). Additionally direct analysis of morphologic features themselves (nucleus/cytoplasm ration and low-complexity morphology) by image analysis can distinguish between resting, self-protected stem cells (FIG. 5) and larger, mitotically active progenitor cells. MDR transporter expression and activity are quantified in both populations by detection of the specific transporter proteins, as discussed herein, and the transport of fluorescent substrates, respectively.

Furthermore, the inventive MDR cancer stem cell preferably is constitutively protected by MDR transporters. The key transporters which are practical for clinical relevance are ABCG2 (mitoxantrone resistance, Breast Cancer Resistance Protein 1), ABCB1 (MDR1, P-glycoprotein), ABCC1 (Multiple Resistance Protein) and Lung Resistance Protein (LRP). Such protection can be assayed as described herein.

Typically, the inventive MDR cancer stem cell is one or more of CD45−, CD90+, CD117+, CD133+, and expresses ABCG2. These cellular markers can be ascertained though standard immunohistochemical methods using monoclonal antibodies that target the respective proteins. Also, typically, the inventive MDR cancer stem cell excludes either rhodamine 123 or Hoechst 33342, and most preferably both dyes. The inventive MDR cancer stem cell also frequently excludes other substrates of MDR transporters, such as those discussed above.

As noted, the MDR stem cell typically is quiescent; however, depending on the culture conditions, the MDR stem cell may be induced to proliferate. Thus, the isolated stem cell can be alone or in a culture of MDR cancer stem cells. In this respect, the invention provides a population comprising one or more MDR cancer stem cells. Where there are more than one cells in the population, the population preferably is substantially homogenous. By “substantially homologous,” in this context, it is meant that a majority of the cells in the population contain the same staining/dye exclusion profile as set forth above. In some embodiments, the population is clonal, such as being descended from a common stem cell. Also, the population can be clonogenic, such that it can establish a clonal population of cells. The population can be maintained in culture in vitro or exist within an animal other than the species of the MDR cancer stem cell population (e.g., a population of human MDR cancer stem cells can exist within an immunocompromized xenograft animal model).

Where the population of one or more MDR cancer stem cell(s) is in vitro, the invention provides a method of assaying for the presence of a target molecule on the surface of a cancer stem cell. In accordance with this method, the population of cancer stem cells is exposed to a ligand recognizing the target molecule under conditions for the ligand to specifically bind the target molecule. Thereafter, the population is assayed for ligand-binding events. The target molecule can be, for example, a receptor, such as a hormone or growth factor receptor (e.g., FGFR, PDGFR, etc.). Alternatively, the target molecule can be an antigenic determinant. The ligand, thus, can be any molecule that specifically binds a receptor, such as an antibody or functional portion thereof (e.g., fab fragment, etc.). Alternatively, a ligand can be a hormone (e.g., growth factor) or portion thereof. Assaying for the ligand-binding event can be achieved by standard methods (e.g.; immunohistochemistry).

Where the population of one or more MDR cancer stem cell(s) is in vitro, the invention provides a method of screening compounds for their potential to kill or inhibit proliferation of MDR cancer stem cells or to cause the cells to lose multi drug resistance. The test compound can be, for example, a small molecule, protein or polypeptide, or nucleic acid.

In accordance with such method a test population of MDR cancer stem cell(s) is cultured and exposed to the test compound. After exposure to the test compound, the population is assayed to ascertain if the test compound kills the cell(s) within the population or retards proliferation (e.g., blocks response to pro-proliferation stimuli). Also, the population can be assayed to determine whether exposure to the test compound has caused the population not to exhibit the MDR cancer stem cell profile (i.e., not CD90+, not CD117+, not CD133+, and not expressing a marker of multiple drug resistance (e.g., ABCB1), and not excluding either rhodamine 123 or Hoechst 33342). The ability of the test compound either to kill the MDR cancer stem cells, inhibit proliferation sensitize to other compounds by MDR inhibition or inactivation (this could be a chemical mediator or a physical mechanism such as heat or radio frequency), or to change the phenotypic profile of the test population away from the MDR cancer stem cell phenotype identifies the test compound as a potential agent for targeting MDR cancer stem cells. Such compounds or procedures are candidates for further development as anti-cancer agents.

In carrying out this method, preferably a control population of MDR cancer stem cell(s) also is maintained, and is treated identically as the test population with the exception of not being exposed to the test compound. Also, preferably a plurality of test populations is employed, such as each population being cultured in a separate well of a multi-well culture plate. This facilitates employing a high-throughput assay system for screening test compounds. For example, the assay can be employed to screen a plurality of test compounds and conditions concurrently. Thus, separate test populations among the plurality of populations is/are exposed to a distinct test compound or to different concentrations of the same test compound or different physical conditions, such as elevated temperature. In this way, multiple compounds can be screened quickly and rapidly using a high throughput assay.

### EXAMPLE 1

This example demonstrates the isolation and identification of MDR cancer stem cells.

A biopsy is obtained from a tumor or normal tissue of a human patient. From the biopsy, single cells are stained with dye-conjugated monoclonal antibodies (CD45, CD44, CD90, CD117, CD133, and ABCG2) for identification (or purification) by flow cytometry. Stained cells are cultured in the presence of fluorescent MDR substrates Rhodamine 123 and Hoechst 33342 for 15-90 min. A viability dye (propidium iodide, DAPI, 7AAD) is added immediately prior to flow cytometry. The population of interest is identified by the following criteria: 1) Live (propidium iodide excluding); 2) Singlet (by forward light scatter pulse analysis; 3) Non-hematopoietic (CD45 negative); 4) CD44+; 5) CD90 or CD117 positive; 6) MDR expression and/or activity by the following criteria: ABCG2+; Rhodamine 123 or Hoechst 33342 transport.

The inventive method has been employed to identify MDR cancer stem cells in over 100 solid tumors: lung cancer 31, esophagus 6, ovarian 3, pleural effusions 39 (small cell lung cancer, non-small cell lung cancer, breast, ovarian, gastric, colon, prostate, renal, pancreatic, melanoma), ovarian ascites 18.

Occasionally, in xenograft assays, grossly non-malignant tissue growth (e.g., breast tissue) has been observed from implantation of tumor-derived tissue. Such results are possibly due to the persistence of non-cancerous stem cells within tumors, which can respond to host environmental conditions to differentiate into tissue, such as breast tissue. Alternatively, cancer stem cells may be induced to form normal appearing tissues by epigenetic reprogramming mediated by the host environment.

### EXAMPLE 2

This example demonstrates an application of the inventive method for identification and isolation of cancer stem cells with constitutive drug resistance.

Tissue Procurement. Freshly isolated tumor and normal tissue specimens are transported to the laboratory immediately after surgical excision for processing.

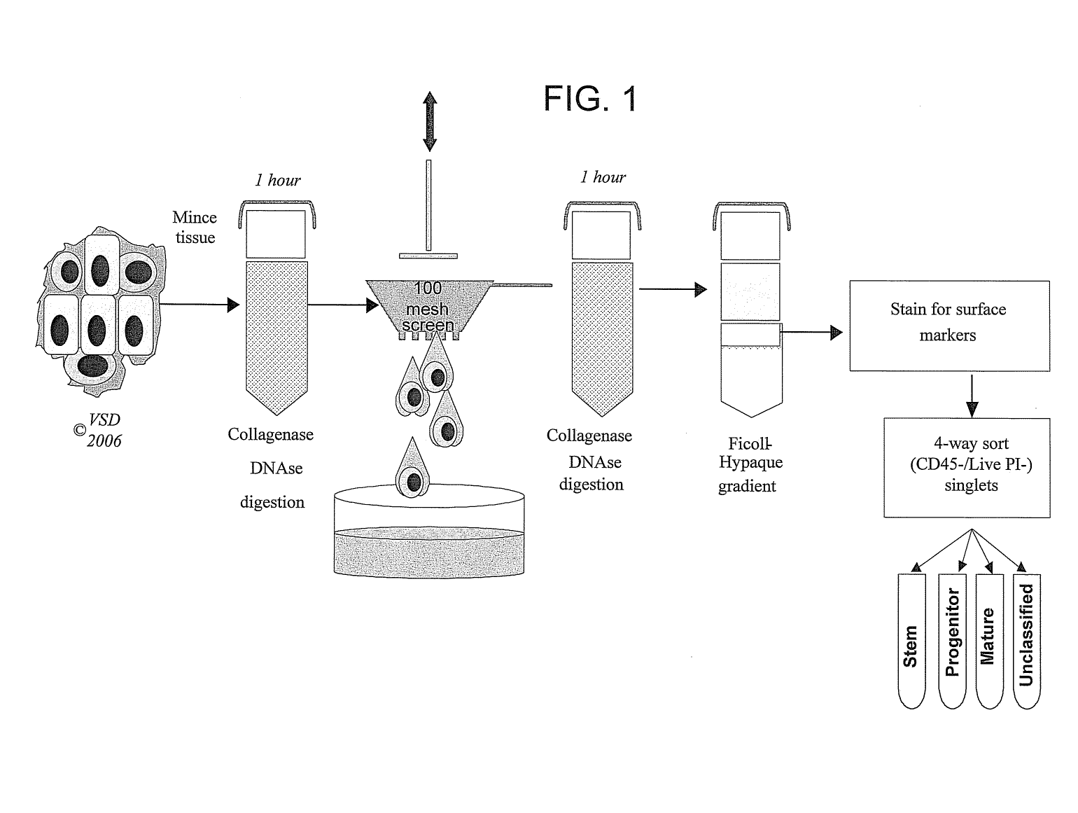

Tissue Digestion. Solid tissues are minced and digested with collagenase and disaggregated through 100 mesh stainless steel screens (FIG. 1) or alternatively by mechanical means alone. Between about 10-500 million viable cells can be isolated from a 5-10 mm³ specimen of tumor or normal lung parenchyma. Pleural effusions and ascites are concentrated, collagenase digested and separated on a ficoll/hypaque gradient. Cells also can be cryopreserved, for example, in medium containing 20% serum and 10% DMSO and held in liquid nitrogen. Disaggregated tissue cells can withstand such cryopreservation with no detectable loss of clonogenicity.

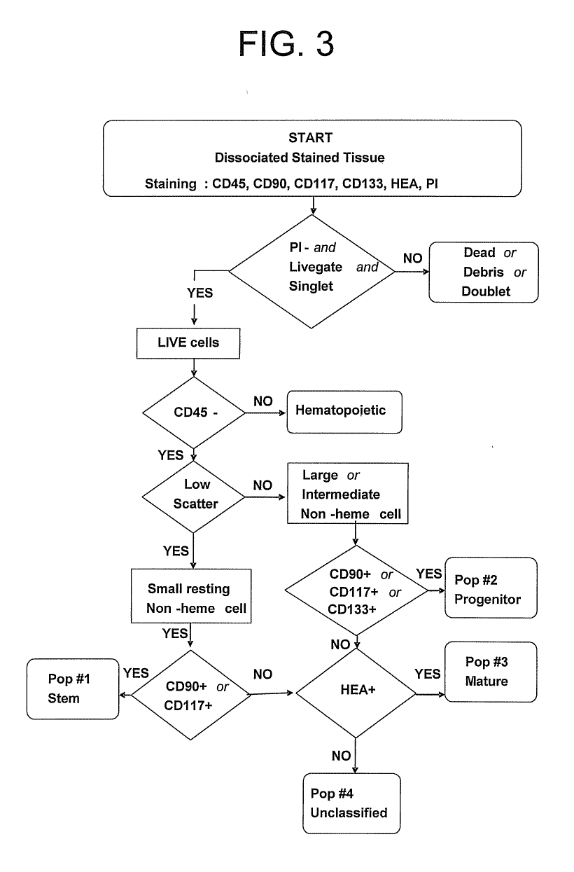

Staining and flow cytometry. The procedure for staining single cell suspensions for flow cytometry has been described in detail (Donnenberg V S, Donnenberg A D., Frontiers in Bioscience, 8:1175-1180, 2003). Five minutes prior to staining with fluorochrome-conjugated monoclonal antibodies, neat mouse serum is added to minimize non-specific binding. Five to 9-color analyses can be performed with any modern commercial flow cytometer (e.g. Dalco-Cytomation CyAn). The cell population of interest can be purified using a commercial fluorescence activated cell sorter (e.g. Dako-Cytomation MoFlo). Samples from each patient group are analyzed, according to principles of rare event detection that have been applied in other contexts (Donnenberg V S, Burckart G J, Griffith B P, Jain A B, Zeevi A, Donnenberg A D. P-glycoprotein (P-gp) is Upregulated in Peripheral T-Cell Subsets from Solid Organ Transplant Recipients. Journal of Clinical Pharmacology, 2001; 41:1271-1279; Donnenberg V S, Donnenberg A D. Identification, rare-event detection and analysis of dendritic cell subsets in broncho-alveolar lavage fluid and peripheral blood by flow cytometry. Frontiers in Bioscience, 8:1175-1180, 2003.; Donnenberg V S, Donnenberg A D, Thompson A W, Zeevi A, Burckart G J, Calhoun W J. In Vivo Maturation of Lung Dendritic Cells from BAL Following Segmental Antigen Challenge (SAC) in Asthmatic Patients. American Respiratory Alliance of Western Pennsylvania. The World Asthma Meeting, 2001; Donnenberg A D, V S Donnenberg, H Shen. Rare-Event Detection and Analysis in Flow Cytometry. The Connection 5: 4-5, 20-21, 2003.) where: 1) A total of 5-10 million cells will be acquired; 2) A basic panel of antigens used for the identification and isolation of stem and progenitor populations from normal epithelial tissues and tumors for 8 and 9 color cytometry is shown in Table 1. The isolation schema is shown in FIG. 3.

Normal epithelial adult tissue stem cells have been identified by anatomical location (i.e. bulge cells in follicle, see Alonso L, Fuchs E. Stem cells of the skin epithelium. PNAS 100 (suppl 1): 11830-11835, 2003., Blanpain, C., Lowry, W. E., Geoghegan, A., Polak, L., and Fuchs, E. Self-renewal, multipotency, and the existence of two cell populations within an epithelial stem cell niche. Cell 118, 635-648, 2004.), morphology (small cytoplasm, smooth nuclear features, synthesis of proteins characteristic of developmental stage (Table 2) and finally functional networks of gene expression by array analysis.

Preferably, proteins characteristic of developmental stages preceding and subsequent to the normal epithelial stem cell stage in normal epithelial stem cells isolated by fluorescence sorting are used to confirm stem cell stage with antigens and antibodies that have been validated in the literature using immunofluorescence microscopy, which is capable of resolving morphology and tissue structures arising in culture. Included in this analysis are markers for earlier developmental stages, especially neurectodermal markers that have been identified in rare cells in the epithelial stem cell population in hair follicle bulges of ectodermal origin (Zhao, X., Das, A. V., Thoreson, W. B., James, J., Wattnem, T. E., Rodriguez-Sierra, J., and Ahmad, I., Adult corneal limbal epithelium: a model for studying neural potential of non-neural stem cells/progenitors. Dev Biol 250, 317-331, 2002; Amoh, Y., Li, L., Katsuoka, K., Penman, S., and Hoffinan, R. M. Multipotent nestin-positive, keratin-negative hair-follicle bulge stem cells can form neurons. PNAS 102, 5530-5534, 2005), as well as airway epithelium of mesenchymal origin. Also, markers of later developmental stages including progenitors and mature epithelium are evaluated. These markers have been chosen for their capacity to discriminate developmental stage in adult epithelium.

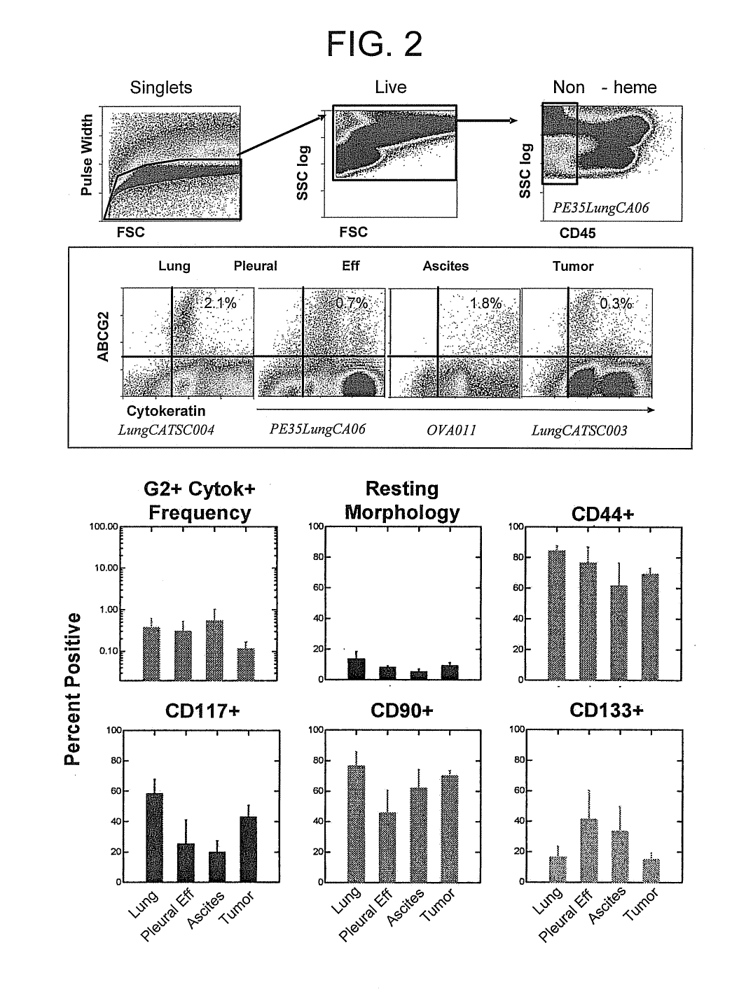

Based on the foregoing: 1) Epithelial stem cells from non-tumor tissue will stage predominantly as epithelial stem cells, with neuroectodermal markers in some cells and; 2) Cancer stem cells also will include expression that is inappropriate for a single developmental stage, both more primitive and more mature. Evidence for the latter is seen in bright cytokeratin expression on the ABCG2+ cells of lung tumors, but not adjacent parenchyma in FIG. 2. Such expression patterns are unique to cancer cells, which have been shown to express primitive markers, including the pluripotency marker, Oct-4, as a result of dysregulation of epigenetic silencing (Galli, R., Binda, E., Orfanelli, U., Cipelletti, B., Gritti, A., De Vitis, S., Fiocco, R., Foroni, C., Dimeco, F., and Vescovi, A. Isolation and Characterization of Tumorigenic, Stem-like Neural Precursors from Human Glioblastoma. Cancer Res 64, 7011-7021, 2004.; Tai, M.-H., Chang, C.-C., Olson, L. K., and Trosko, J. E. Oct4 expression in adult human stem cells: evidence in support of the stem cell theory of carcinogenesis. Carcinogenesis 26, 495-502, 2005). Analysis for inappropriate expression or asynchronous expression of developmental cell surface markers provides a simple flow cytometric screening tool to distinguish between normal and cancer stem cells.

Flow cytometric cell sorting. Sorting is performed on the classification parameters shown in FIG. 3, or alternatively on MDR markers as shown in Table 1. Cells with stem (small resting, high nucleus/cytoplasm ratio, CD117+, CD90+, CD133+ MDR+) and progenitor features (large, low nucleus/cytoplasm ratio, some cell in cycle, CD117+ or CD09+ or CD133+, MDR−) can be found in disaggregated normal and neoplastic lung at a low frequency (<0.1%). Despite the similar phenotype with respect to this restricted set of markers, sorted tumorigenic stem cells give rise to tumors in SCID/NOD mice and NS do not, proving that they are not simply normal tissue stem cells infiltrating the tumor.

In vitro growth of tumorogenic and normal tissue stem cell expansion. Initial cell cultures (300 sorted CFDA-SE labeled cells/well) are conducted in 24-well plates on a monolayer of heavily irradiated (100 Gy) primary mouse embryonic fibroblasts under conditions optimized for growth of embryonic stem cells (3% O₂, 5% CO₂, DMEM with 15% fetal calf serum, buffalo rat liver conditioned medium, non-essential amino acids, (-mercaptoethanol and recombinant leukemia inhibitory factor) (Niedernhofer L J. Odijk H. Budzowska M. van Drunen E. Maas A. Theil A F. de Wit J. Jaspers N G. Beverloo H B. Hoeijmakers J H. Kanaar R. The structure-specific endonuclease Ercc1-Xpf is required to resolve DNA interstrand cross-link-induced double-strand breaks. Molecular & Cellular Biology. 24(13):5776-87, 2004). Additionally, a commercially available tissue culture medium for human embryonic stem cell use can be substituted. After two weeks in culture a >100-fold expansion in cell number can be obtained.

### EXAMPLE 3

In this Example, the following definitions and abbreviations are employed:
putative tumor stem cell (TS),
tumor progenitor cell (TP),
mature tumor (TM) cell populations,
normal epithelial stem (NS),
normal progenitor (NP), and
normal mature (NM) cells.
“Stem cell” indicates a cell with a normally resting state (G₀), and the properties of self-protection and self-renewal (high capacity for serial passage).
A “progenitor cell” is understood to be the immediate progeny of the stem cell and have high proliferative capacity, but exhibits no self-protection and little self-renewal (cannot be serially passaged).
“Mature cells” are those that are unable to proliferate. This paradigm is not universal, but appears to hold in normal epithelial tissue (Alonso L, Fuchs E. Stem cells of the skin epithelium. PNAS 100 (suppl 1): 11830-11835, 2003).

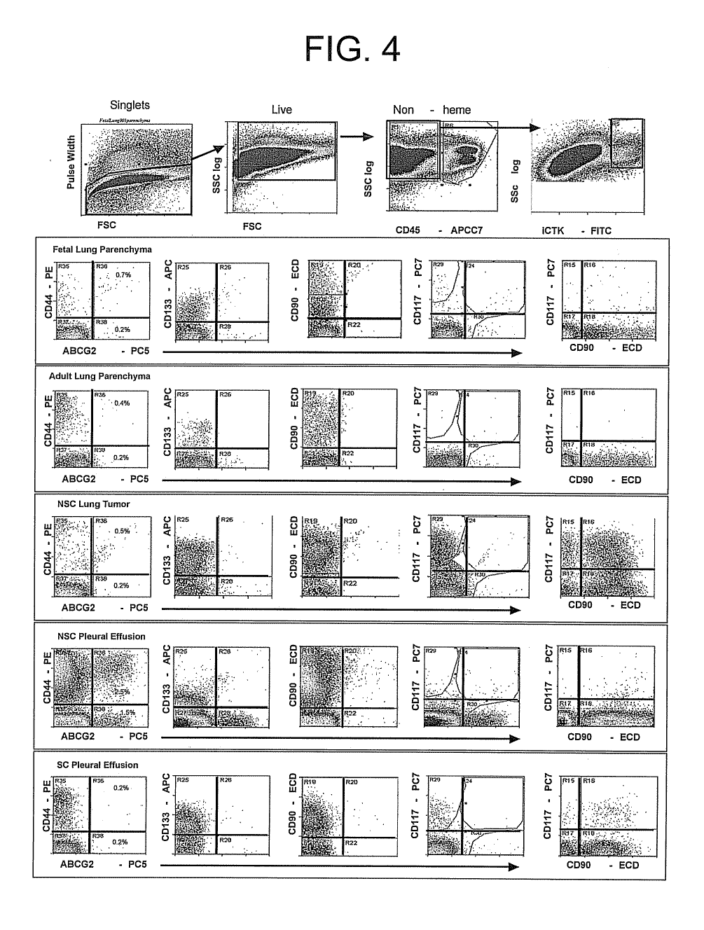

Detection of stem and progenitor cells in normal fetal and adult lung parenchyma, tumor and malignant effusions. In order to detect putative lung stem and amplifying progenitor populations, lung fetal, adult normal lung, and freshly isolated non-small cell (NSC) lung tumor were minced and collagenase digested. Additionally two malignant effusions (NSC and SC lung cancer) were collagenase digested. Viable cells were isolated by Ficoll/Hypaque gradient centrifugation and stained with a cocktail of antibodies designed to detected non-hematopoietic (CD45−), cytokeratin (CK) dim or bright epithelial stem cells (FIG. 4). The gating strategy used for all samples is illustrated in the top panel (fetal lung, 18 weeks gestation). Cell clusters are eliminated (FSC by FSC Pulse width), apoptotic cells and debris are eliminated (FSC by SSC log) and CD45− intracellular cytokeratin+ cells are identified. Subsequent analyses are performed on this population. For identification of putative epithelial stem and progenitor populations, stem/progenitor markers previously described in hematopoietic cells were the primary focus, which preliminary experiments indicated also are expressed on rare subsets of CD45− human epithelial antigen (HEA)+ cells. In contrast to normal adult lung, fetal lung cells evidenced a unique population of CD90 dim cells representing 24% of gated (CD45−/CK+) cells. These cells are large and uniformly CD117− and CD133+ (not shown) and represent the best candidate for the amplifying progenitor cell engaged in organogenesis. In fetal lung ABCG2+ cells (putative resting stem cells) represented 0.9% of gated cells (n=3). These were present at approximately the same frequency in adult lung. The expression of stem cell markers in NSC lung tumors is more variable from patient to patient (n=6, not shown). In the present example there is a prominent population of CD90+ CD133+ cells, but unlike those in fetal lung, they are CD117+. Examples of malignant effusions from NSC and SC lung cancer (n=28) show prominent populations of ABCG2+ cells, and fetal-like CD90 dim cells, respectively. Analysis was restricted to CK+ cells in order to assure that cells were epithelial in origin. CD45− CK− progenitor and stem cells were also seen in all tissues and represent a separate analysis (not shown).

These data demonstrate the successful sorting of stem and progenitor populations from cryopreserved tissues/fetal-like CD90 dim cells, respectively. Analysis was restricted to CK+ cells in order to assure that cells were epithelial in origin. CD45− CK− progenitor and stem cells were also seen in all tissues and represent a separate analysis (not shown).

To investigate expression of the MDR transporter ABCG2 in freshly isolated therapy naïve tumor cells, single cell suspensions were prepared from solid tumors (lung Ca=7, ovarian=3) and malignant ascites and effusions (lung=2, ovarian=6, gastric=1) by mechanical dissection and collagenase digestion. Samples were stained by 7-color flow cytometry for expression of ABCG2, CD45, intracellular cytokeratin, CD44, CD90, CD117 and CD133. An average of 2.5 million events were acquired for each sample (min=200,000, max=6,000,000). All newly diagnosed untreated epithelial tumors contained a rare subset of CD45−/cytokeratin dim/ABCG2+ cells (0.43±0.57 of CD45− cells). ABCG2+ cytokeratin dim cells also expressed CD44 (69±18%), and the stem/progenitor markers CD90 (62±20%), CD117 (34±23%) and CD133 (25±23%). Eight±5% of ABCG2+ cells (0.03% of CD45− cytokeratin dim) had low light scatter profiles compatible with small resting morphology. These data demonstrate that therapy naïve tumors contain a rare subpopulation of cells that constitutively express the MDR transporter ABCG2, and therefore presumptively have a priori chemoresistance. A proportion of these cells are tumorigenic in a xenograft model system and clonogenic in vitro (see below). Such cancer stem cells are consistent with a model of being constitutively MDR active and are the likely reservoir subpopulation of drug-resistant cells that lead to relapse after apparently successful initial therapy.

Freshly isolated CD45− CD90+ tumorigenic cells have a small resting morphology. The sample analyzed here (FIG. 5) on the Amnis ImageStream imaging flow cytometer was also sorted and injected into SCID/NOD mice. CD45− CD90+ cells had a unique small resting morphology with a high nucleus to cytoplasm ratio (Panel A). Three groups of 3 mice each were injected i.v. with 3 different cell populations: 1) Stem1: CD45− CD90+ CD133− HEA− (20,000 cells/mouse); 2) Stem2: CD45− CD90+ CD133+ HEA+ (15,000 cells/mouse); 3) Unsorted tumor (15,000 cells/mouse). As of Mar. 30, 2006 (day 205) one mouse (unsorted group) died of a thymoma, and one mouse (Stem2) was sacrificed with multiple tumor nodules in the lungs.

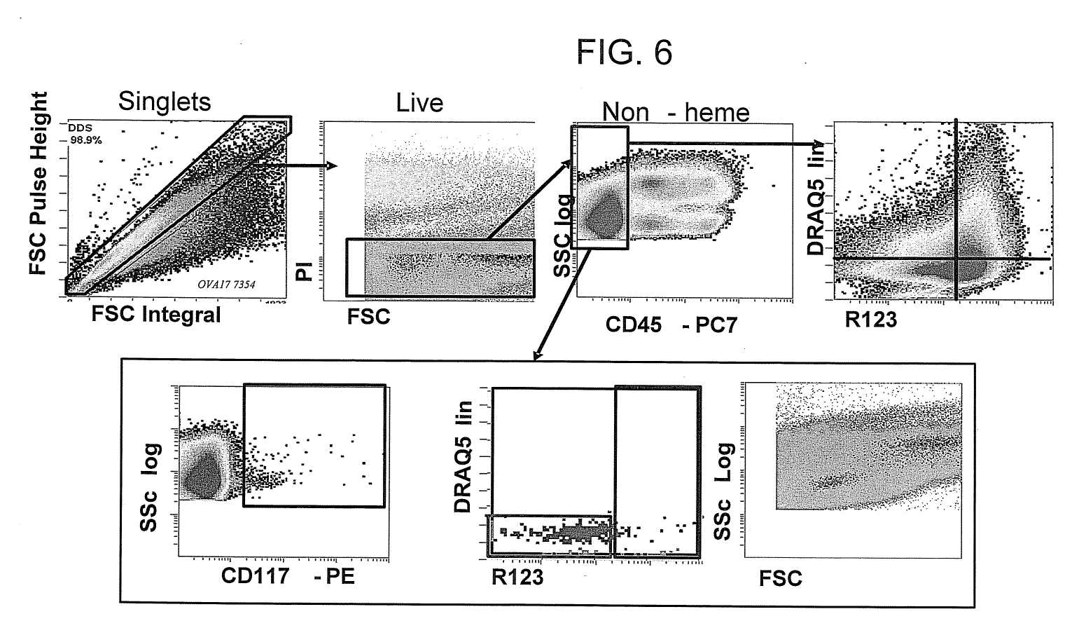

MDR activity in malignant effusions is limited to a CD117 (c-kit)+ population of small resting cells. Dye efflux provides a sensitive and specific assay for MDR activity. The data depicted in FIG. 6 demonstrate that in the well defined CD117+ (stem cell factor receptor+) population a malignant ascites, MDR activity is limited to cells with low forward and side scatter. This population comprised 0.3% of nucleated cells in the ascites. R123 efflux was abrogated by the MDR competitive inhibitor cyclosporine, confirming the assay specificity. Accordingly, the R123 efflux assay can be used in conjunction with multiparameter immuno-phenotypic characterization to identify putative lung stem cell populations.

Self-renewal and differentiation of tumor stem cells in culture under ES conditions. Distinguishing between non-clonogenic, clonogenic and self-renewing tumor cells is essential to the biological characterization of tumor subsets to be described here. There is an extensive literature on detection of tumor “colonies” in short-term culture, but these represent progenitor cells of limited replicative potential. In experiments with long-term growth of sorted CD90+ tumor cells using a variety of media, growth factors and substrates, growth was extremely slow (limit dilution cultures were scored at 8 weeks in culture) and expansion was limited.

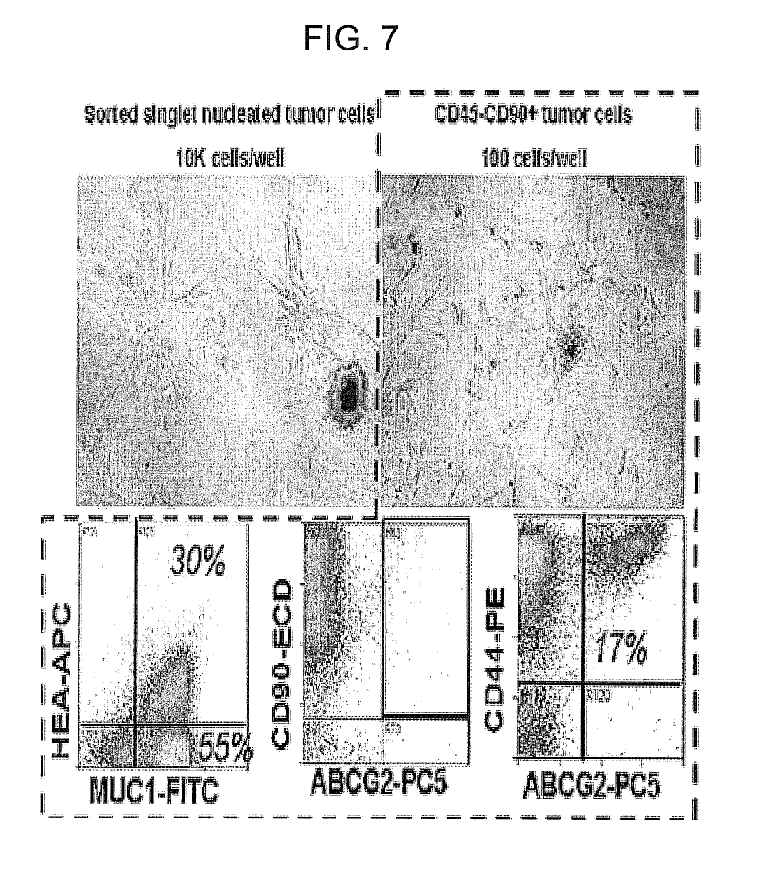

A culture system has been developed that maximizes expansion and self-renewal of candidate tumor stem and progenitor populations by capitalizing on a system originally optimized for the growth of murine embryonic stem cells. As an example, freshly isolated non-small cell primary lung tumor cell suspensions were sorted for: 1) Total viable nucleated cells (10,000 cells/well); 2) CD45− CD90+ (30-300 cells/well); and 3) CD45− ABCG2+ (30-300 cells/well). Cells were sorted directly into flat bottom 96-well plates coated with either a monolayer of irradiated mouse embryonic fibroblasts (MEFs) or 0.4% gelatin. Cultures were held at low oxygen tension at 37° C. All cell populations gave rise to colonies after about 2 weeks in culture. Total tumor and the CD90+ population gave rise to structures resembling embryoid bodies (FIG. 7), whereas fibroblasts and colonies of round cells grew out of the non-hematopoietic ABCG2+ population in the 2-week time interval. Cultures were split at 21 days and were passaged on irradiated MEF monolayers. Cells at passage 4 were harvested for flow cytometry. A cell cluster resembling an embryoid body was harvested for transmission electron microscopy (EM). Despite the enormous expansion over 4 passages, 17% of cultured CD45− CD90+ cells retained this stem-like phenotype, indicating self-renewal and expansion (FIG. 7). The bulk of cells, however, lost stem cell markers, expressed differentiation antigens (HEA, MUC-1) and showed increased size and granularity, consistent with differentiation. EM on the cell cluster was consistent with an embryoid body, having interior cells with smooth chromatin and pleomorphic shapes characteristic of early stem cells, and squamous epithelial-like outer cells with closely opposed junctions (not shown).

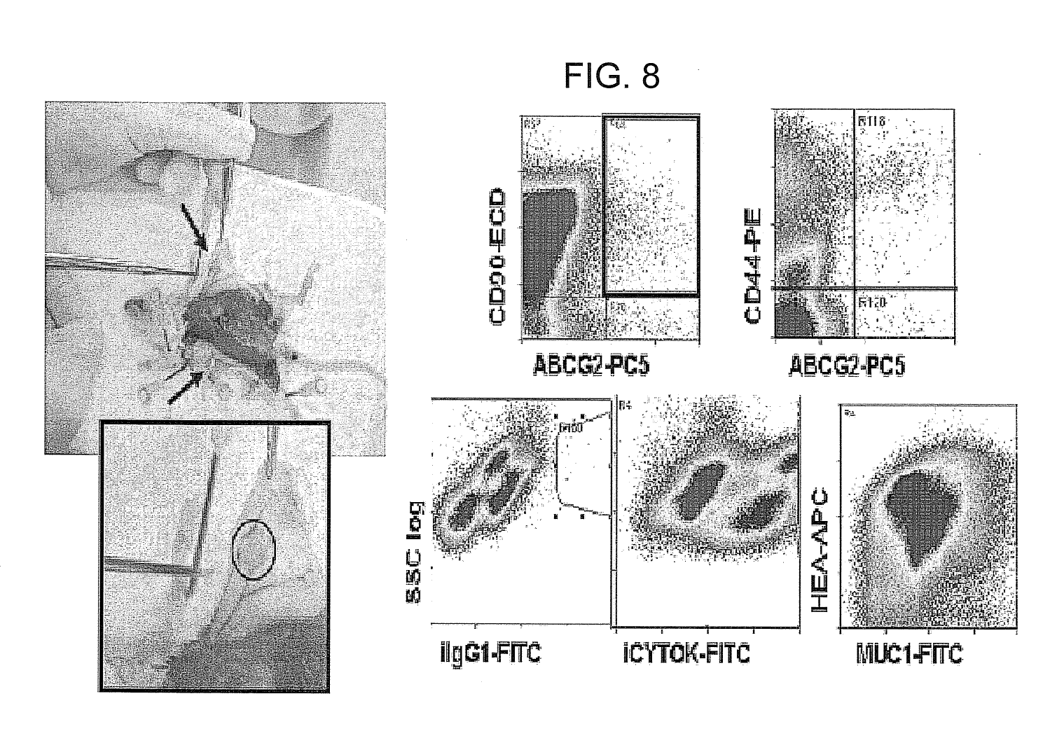

The rare CD45− CD90+ tumor stem cell is tumorigenic at high frequency. In an experiment performed prior to our adoption of ES culture methods, data have shown that CD45− CD90+ sorted tumor cells (cryopreserved pleural effusion) were clonogenic in an 8-week limit dilution assay at 30 cells/well. At the time that these cultures were established, experiments also tested their tumorogenicity in SCID/NOD mice (FIG. 8). Populations of sorted CD45− cells were tested: 1) CD90+ ABCG2+ (40 cells/site, 5 mice, 1 injection site; and 600 cells/site, 5 mice, 1 injection site); 2) CD90+ ABCG2− (60 cells/site, 5 mice, 1 injection site; and 10,000 cells/site, 5 mice, 1 injection site). Additionally, two mice were injected (10,000 cells/site, 2 mice, 2 injection sites) with: 1) unseparated tumor cells, or 2) irradiated (10,000 rads) unseparated tumor cells. All sorted populations were mixed with 10,000 irradiated (10,000 rad) unsorted tumor cells, suspended in matrigel and 30% clarified effusion fluid, and injected subcutaneously (mammary and inguinal fat pads).

Of the 5 mice injected with CD90+ cells, 1 died without human tumor 5 months after injection. The 4 remaining mice grew tumors at 3 or 4 sites/mouse from both the CD90+ ABCG2− and CD90+ ABCG2+ populations. Tumors were first palpable 5-12 months after injection. Tumors were of human origin and contained, as a rare population (0.63% of CD45− cells), cells of the original stem-like phenotype (CD45− CD90+), indicating self-renewal. The majority of CD45− cells (˜70%) were mature tumor cells which co-expressed MUC-1 and HEA and had high forward and side scatter. One mouse injected with 10,000 unsorted tumor cells developed a small tumor (½ sites) at 12 months. None of the 5 mice injected with CD44+ CD90− cells developed a tumor (all died spontaneously between day 116 and 320). Spontaneous deaths were due to murine tumors/thymomas, to which SCID/NOD mice are predisposed (Prochazlca M, Gaskins H R, Shultz L D, Leiter E H. The nonobese diabetic scid mouse: Model for spontaneous thymomagenesis associated with immunodeficiency (severe combined immunodeficiency mutation). Proc. Natl. Acad. Sci.-USA Vol. 89, pp. 3290-3294, 1992).

Additional experiments in progress with >10 months followup, testing different injection sites (i.v. vs. sc, small cell vs. non-small cell lung cancer), have yielded tumors in SCID/NOD mice injected with CD45− CD90+ sorted therapy naïve lung tumor cells. The first serial transfer study (using CD90+ cells resorted from the tumor shown in FIG. 8) yielded its first tumor 4 months after transfer. Despite the small sample size, these experiments demonstrate that the CD45−CD90+ fraction contains cells that are tumorigenic at very high frequency. Their immunophenotype, self-renewal and differentiation in culture, and small resting morphology qualify them as cancer stem cells.

Relationship between MDR activity and cell cycle. Even in tumors with a high mitotic index, MDR activity is restricted to resting cells. This can be seen in FIG. 6 (aggressive malignant ascites), where all of the CD45− CD117+ MDR active cells (red dots) are out of cycle (Draq5 low). This observation also is in agreement with our published work in T-cell subsets (Donnenberg V S. Burckart G J. Zeevi A. Griffith B P. Iacono A. McCurry K R. Wilson J W. Donnenberg A D. P-glycoprotein activity is decreased in CD4+ but not CD8+ lung allograft-infiltrating T cells during acute cellular rejection. Transplantation. 77(11):1699-706, 2004). Although naïve T cells are not stem cells, they self-renew and use MDR as a self-protective mechanism. The salient feature is that MDR activity is restricted to resting cells and is rapidly lost as T-cells become activated by the mitogen SEB. An identical strategy can determine whether resting adult tissue stem cells (normal and neoplastic), down-regulate their constitutively high MDR activity when induced into cycle under ES conditions.

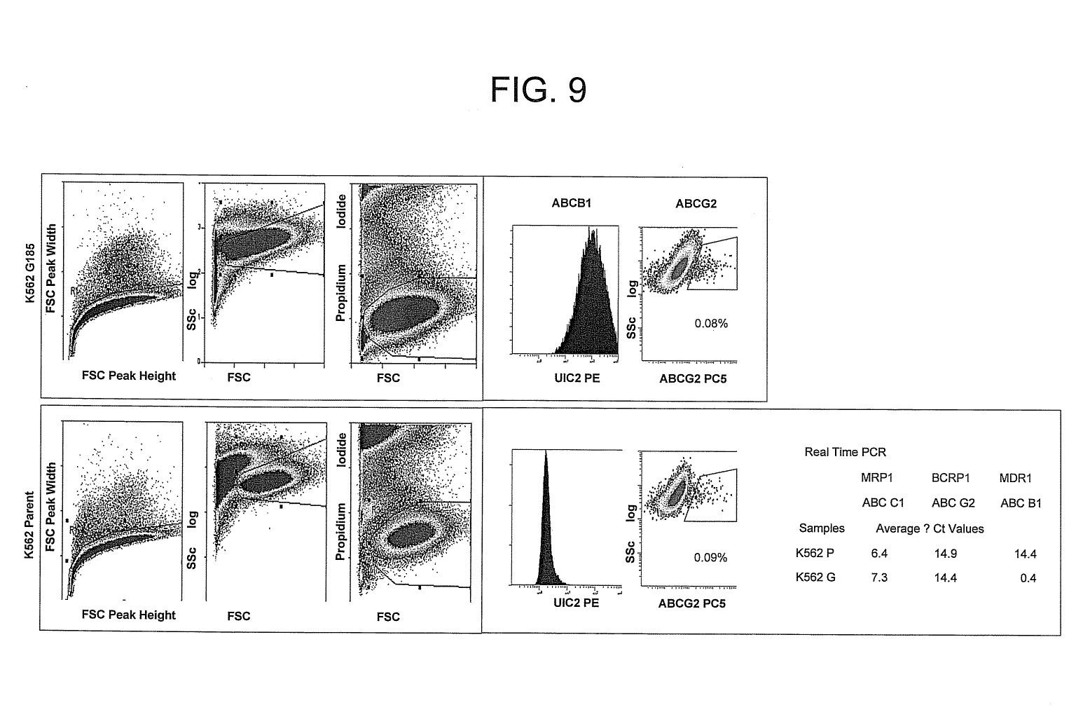

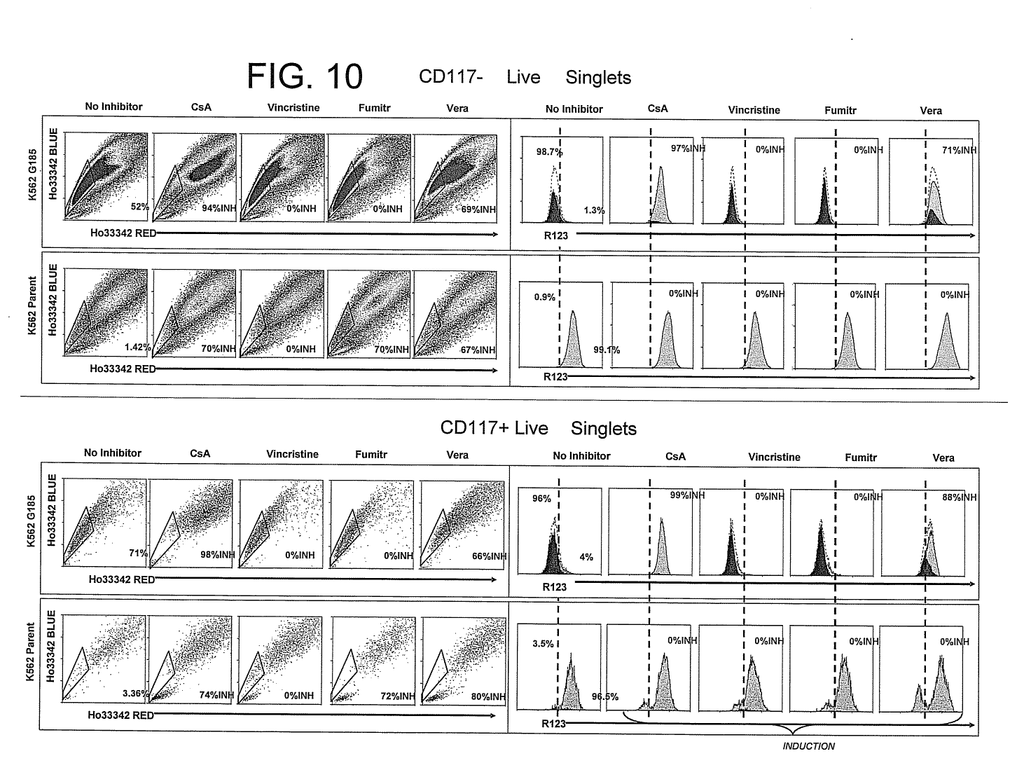

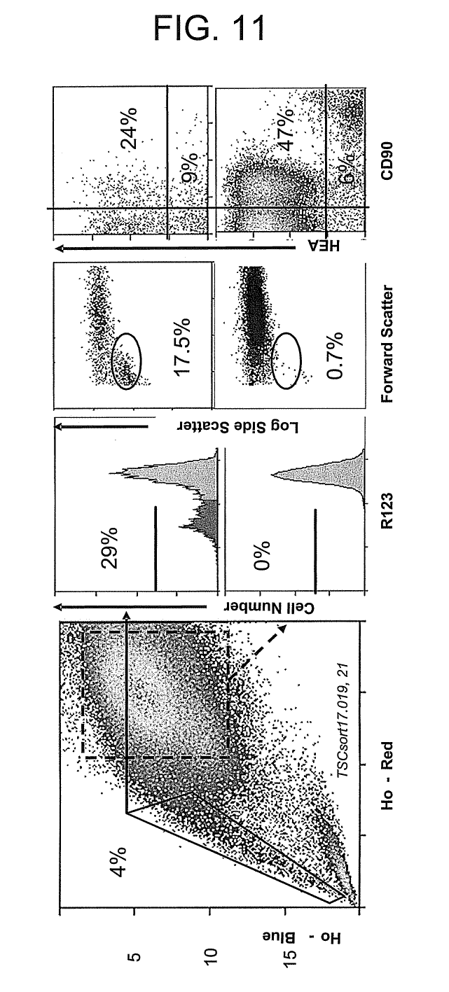

Simultaneous measurement of R123 and Ho33342 dye efflux. FIG. 10 shows a complex multi-outcome experiment in which demonstrates the ABCG2 specificity of the inhibitor fumitremorgin on R123 and Ho33342 transport, in a rare subpopulation detected in two different cell lines. These data measures efflux of both dyes simultaneously, and demonstrate that the Ho333342dim phenotype (side population) associated with adult tissue stem cells is identical to the R123dull population first described in primitive hematopoietic stem cells (Udomsakdi C, Eaves C J, Sutherland H J, Lansdorp P M. Separation of functionally distinct subpopulations of primitive human hematopoietic cells using rhodamine-123. Exp Hematol. (5):338-42, 1991). Two cell lines were used, the parent line K562, which has a small subpopulation of CD117+ cells which constitutively express ABCG2 (BCRP1), and the ABCB1 (MDR1) transfectant line K562-G185 (39) (FIG. 9). G185 cells were maintained in vincristine (100 ng/mL) to select against ABCB1 negative cells. Real time PCR confirmed the specificity of the flow cytometric determinations, showing that ABCB1 expression is limited to the transfected G185 cell line. FIG. 10 demonstrates that both Ho33342 and R123 are transported by ABCB1. Therefore, our ability to functionally discriminate between these transporters depends upon the use of the ABCG2-specific inhibitor fumitremorgin. This can be seen in FIG. 10, where fumitremorgin abrogates Ho33342 transport the CD117+ population of the parental K562 cells, but not R123 transport in the G185 transfectant. This shows that for the proposed studies using freshly isolated tumor cells, the Ho33342 and R123 efflux assays can be used interchangeably, depending on the fluorochrome combinations required (Ho33342 requires a u.v. laser but does not occupy the green emissions channel required for the tracing dye CFDA-SE). The ABCG2-specific inhibitor fumitremorgin, and the potent ABCG2 and ABCB1 dual inhibitor cyclosporine, will permit us to measure functional resistance in rare subpopulations like the CD117+ subset shown here. Similarly, the specific antibodies anti-ABCG2 and UIC2 (anti-ABCB1) (FIG. 9) quantification of expression in small subpopulations of freshly isolated samples.

### EXAMPLE 4

This example demonstrates the presence of ABCG2 and ABCB 1 activity in freshly isolated therapy naïve non-small cell lung cancer.

Antibody stained suspended tumor cells were incubated simultaneously with the ABCG2/ABCB1 substrate Hoechst 33342 (8 microM) plus the ABCB1 substrate rhodamine 123 (R123, 0.13 microM) for 90 minutes at 37° C. Propidium iodide (PI, 10 microg/mL) was added immediately before sample acquisition. All events were gated on PI excluding (live), non-hematopoietic singlets. Five million events were collected. This basic experimental design has been repeated, with modifications, on 10 samples from untreated breast, ovarian, gastric and lung tumors with consistent results.

Data from this experiment are presented in FIG. 11. The leftmost panel shows a small population (4%) of Hoechst 33342-excluding cells in the typical pattern of the Side Population (SP). SP (top panels) and non-SP cells (bottom panels) were further characterized: A proportion of SP cells also excluded the ABCB1 substrate dye R123. These accounted for 29% of the SP cells (color-evented red in the dot plots) and accounted for almost all of the cells with low forward and side light scatter consistent with a resting morphology (FIG. 5). Non-SP cells did not transport R123 and were exclusively of high light scatter. A significant proportion of both SP and non-SP cells expressed CD90, often in combination with epithelial specific antigen, HEA. Coincubation of tumor cells with Hoechst 33342, R123, and the ABCG2 specific inhibitor fumitremorgin (10 microM) resulted in 75% inhibition of the SP phenotype.

Of the 4% of non-hematopoietic cells expressing the Side Population phenotype, 29% had concurrent R123 efflux, whereas none of the SP negative cells (Hoechst bright) transported rhodamine. The SP population also uniquely contained a subset (17.5%) of cells with low morphological complexity. These small cells accounted for the majority of cells which pumped both Hoechst 33342 and R123 (FIG. 11), color-evented red). The ABC transporter specificity of dye efflux was demonstrated with the ABCG2-specific inhibitor fumitremorgin, which inhibited 75% of the SP phenotype. CD90+ cells were present in both SP+ and SP negative fractions, supporting the idea that a proportion of tumor cells with stem cell markers and resting morphology are constitutively protected by ABCG2. Further, when R123 efflux was examined among the CD45− CD117+ subset of untreated lung, breast, ovarian and gastric cancers, MDR activity was restricted to the subset with low morphologic complexity and G1/G0 cell cycle phase (n=10, data not shown).

### REFERENCES

Alonso L, Fuchs E. Stem cells of the skin epithelium. PNAS 100 (suppl 1): 11830-11835, 2003.
Amoh, Y., Li, L., Katsuoka, K., Penman, S., and Hoffman, R. M. Multipotent nestin-positive, keratin-negative hair-follicle bulge stem cells can form neurons. PNAS USA 102, 5530-5534, 2005.
Appelbaum F R, Fisher L D, Thomas E D. Chemotherapy v marrow transplantation for adults with acute nonlymphocytic leukemia: a five-year follow-up. Blood. 1988;72:179-184.
Baines P, Visser J W. Analysis and separation of murine bone marrow stem cells by H33342 fluorescence-activated cell sorting. Exp Hematol. 1983;11:701-708.
Biedler J L, Riehm H. Cellular resistance to actinomycin D in Chinese hamster cells in vitro: cross-resistance, radioautographic, and cytogenetic studies. Cancer Res. 1970;30:1174-1184.
Blanpain, C., Lowry, W. E., Geoghegan, A., Polak, L., and Fuchs, E. Self-renewal, multipotency, and the existence of two cell populations within an epithelial stem cell niche. Cell 118, 635-648, 2004.
Bunting K D, Zhou S, Lu T, Sorrentino B P. Enforced P-glycoprotein pump function in murine bone marrow cells results in expansion of side population stem cells in vitro and repopulating cells in vivo. Blood. 2000;96:902-909.
Burnett A K. Annotation: current controversies—which patients with acute myeloid leukaemia should receive a bone marrow transplantation? An adult treater's view. Br J Haematol. 2002;118:357-364.
Carrion C, de Madariaga M A, Domingo J C. In vitro cytotoxic study of immunoliposomal doxorubicin targeted to human CD34(+) leukemic cells. Life Sci. 2004;75:313-328.
Chen C C, Meadows B, Regis J, et al. Detection of in vivo P-glycoprotein inhibition by PSC 833 using Tc-99m sestamibi. Clin Cancer Res. 1997;3:545-552.
Dey S, Ramachandra M, Pastan I, Gottesman M M, Ambudkar S V. Evidence for two nonidentical drug-interaction sites in the human P-glycoprotein. Proc Natl Acad Sci U S A. 1997;94:10594-10599.
Donnenberg A D, V S Donnenberg, H Shen. Rare-Event Detection and Analysis in Flow Cytometry. The Connection 5: 4-5, 20-21, 2003.
Donnenberg V S, and Donnenberg A D. Multiple drug resistance in cancer revisited: the cancer stem cell hypothesis. J Clin Pharmacol. 2005 August;45(8):872-7.
Donnenberg V S, Burckart G J, Griffith B P, Jain A B, Zeevi A, Donnenberg A D. P-glycoprotein (P-gp) is Upregulated in Peripheral T-Cell Subsets from Solid Organ Transplant Recipients. Journal of Clinical Pharmacology, 2001; 41:1271-1279.
Donnenberg V S, Donnenberg A D, Thompson A W, Zeevi A, Burckart G J, Calhoun W J. In Vivo Maturation of Lung Dendritic Cells from BAL Following Segmental Antigen Challenge (SAC) in Asthmatic Patients. American Respiratory Alliance of Western Pennsylvania. The World Asthma Meeting, 2001.
Donnenberg V S, Donnenberg A D. Identification, rare-event detection and analysis of dendritic cell subsets in broncho-alveolar lavage fluid and peripheral blood by flow cytometry. Frontiers in Bioscience, 8:1175-1180, 2003.
Donnenberg V S. Burckart G J. Zeevi A. Griffith B P. Iacono A. McCurry K R. Wilson J W. Donnenberg A D. P-glycoprotein activity is decreased in CD4+ but not CD8+ lung allograft-infiltrating T cells during acute cellular rejection. Transplantation. 77(11):1699-706, 2004
Doyle L A, Yang W, Abruzzo L V, et al. A multidrug resistance transporter from human MCF-7 breast cancer cells. Proc Natl Acad Sci USA. 1998;95:15665-15670.
Fiala S. The cancer cell as a stem cell unable to differentiate: a theory of carcinogenesis. Neoplasma. 1968;15:607-622.
Fojo A, Hamilton T C, Young R C, Ozols R F. Multidrug resistance in ovarian cancer. Cancer. 1987;60(suppl 8):2075-2080.
Galli, R., Binda, E., Orfanelli, U., Cipelletti, B., Gritti, A., De Vitis, S., Fiocco, R., Foroni, C., Dimeco, F., and Vescovi, A. Isolation and Characterization of Tumorigenic, Stem-like Neural Precursors from Human Glioblastoma. Cancer Res 64, 7011-7021, 2004;
Giangreco A, Shen H, Reynolds S D, Stripp B R. Molecular phenotype of airway side population cells. Am J Physiol Lung Cell Mol Physiol. 2004;286:L624-L630.
Goodell M A, Brose K, Paradis G, Conner A S, Mulligan R C. Isolation and functional properties of murine hematopoietic stem cells that are replicating in vivo. J Exp Med. 1996;183:1797-1806.
Gros P, Ben Neriah Y B, Croop J M, Housman D E. Isolation and expression of a complementary DNA that confers multidrug resistance. Nature. 1986;323:728-731.
Gros P, Croop J, Roninson I, Varshavslcy A, Housman D E. Isolation and characterization of DNA sequences amplified in multidrug-resistant hamster cells. Proc Natl Acad Sci USA. 1986;83:337-341.
Hamburger A W, Salmon S E. Primary bioassay of human tumor stem cells. Science. 1977;197:461-463.
Harker W G, Slade D L, Dalton W S, Meltzer P S, Trent J M. Multidrug resistance in mitoxantrone-selected HL-60 leukemia cells in the absence of P-glycoprotein overexpression. Cancer Res. 1989;49:4542-4549.
Kessel D, Botterill V, Wodinsky I. Uptake and retention of daunomycin by mouse leukemic cells as factors in drug response. Cancer Res. 1968;28:938-941.
Leonard G D, Fojo T, Bates S E. The role of ABC transporters in clinical practice. Oncologist. 2003;8:411-424.
Ling V, Thompson L H. Reduced permeability in CHO cells as a mechanism of resistance to colchicine. J Cell Physiol. 1974;83(1):103-116.
Lum B L, Fisher G A, Brophy N A, et al. Clinical trials of modulation of multidrug resistance: pharmacolinetic and pharmacodynamic considerations. Cancer. 72(suppl 11):3502-3514.
Merry S, Courtney E R, Fetherston C A, Kaye S B, Freshney R I. Circumvention of drug resistance in human non-small cell lung cancer in vitro by verapamil. Br J Cancer. 1987;56:401-405.
Mulder A H, Visser J W. Separation and functional analysis of bone marrow cells separated by rhodamine-123 fluorescence. Exp Hematol. 1987;15:99-104.
Niedernhofer L J. Odijk H. Budzowska M. van Drunen E. Maas A. Theil A F. de Wit J. Jaspers N G. Beverloo H B. Hoeijmakers J H. Kanaar R. The structure-specific endonuclease Ercc1-Xpf is required to resolve DNA interstrand cross-link-induced double-strand breaks. Molecular & Cellular Biology. 24(13):5776-87, 2004.
Prochazka M, Gaskins H R, Shultz L D, Leiter E H. The nonobese diabetic scid mouse: Model for spontaneous thymomagenesis associated with immunodeficiency (severe combined immunodeficiency mutation). Proc. Natl. Acad. Sci.-USA Vol. 89, pp. 3290-3294, 1992
Reya T, Morrison S J, Clarke M F, Weissman I L. Stem cells, cancer, and cancer stem cells. Nature. 2001;414:105-111.
Roninson I B, Abelson H T, Housman D E, Howell N, Varshavsky A. Amplification of specific DNA sequences correlates with multi-drug resistance in Chinese hamster cells. Nature. 1984;309:626-628.
Roninson I B, Chin J E, Choi K G, et al. Isolation of human mdr DNA sequences amplified in multidrug-resistant KB carcinoma cells. Proc Natl Acad Sci USA. 1986;83:4538-4542.
Rustum Y M, Radel S, Campbell J, Mayhew E. Approaches to overcome in vivo anti-cancer drug resistance. Prog Clin Biol Res. 1986; 223:187-202.
Shoemaker R H, Curt G A, Carney D N. Evidence for multidrug-resistant cells in human tumor cell populations. Cancer Treat Rep. 1983;67:883-888.
Spangrude G J, Heimfeld S, Weissman I L. Purification and characterization of mouse hematopoietic stem cells. Science. 1988;241: 58-62.
Tai, M.-H., Chang, C.-C., Olson, L. K., and Trosko, J. E. Oct4 expression in adult human stem cells: evidence in support of the stem cell theory of carcinogenesis. Carcinogenesis 26, 495-502, 2005.
Tan B, Piwnica-Worms D, Ratner L. Multidrug resistance transporters and modulation. Curr Opin Oncol. 2000;12:450-458.
Twentyman P R, Fox N E, Bleehen N M. Drug resistance in human lung cancer cell lines: cross-resistance studies and effects of the calcium transport blocker, verapamil. Int J Radiat Oncol Biol Phys. 1986;12:1355-1358.
Udomsakdi C, Eaves C J, Sutherland H J, Lansdorp P M. Separation of functionally distinct subpopulations of primitive human hematopoietic cells using rhodamine-123. Exp Hematol. 1991;19:338-342.
Volm M. Multidrug resistance and its reversal. Anticancer Res. 1998;18:2905-2917.
Weiden P L, Flournoy N, Thomas E D, et al. Antileukemic effect of graft-versus-host disease in human recipients of allogeneic-marrow grafts. N Engl J Med. 1979;300:1068-1073.
Zhao, X., Das, A. V., Thoreson, W. B., James, J., Wattnem, T. E., Rodriguez-Sierra, J., and Ahmad, I., Adult corneal limbal epithelium: a model for studying neural potential of non-neural stem cells/progenitors. Dev Biol 250, 317-331, 2002.
Zhou S, Morris J J, Barnes Y, Lan L, Schuetz J D, Sorrentino B P. Bcrp1 gene expression is required for normal numbers of side population stem cells in mice, and confers relative protection to mitoxantrone in hematopoietic cells in vivo. Proc Natl Acad Sci USA. 2002;99:12339-12344.
Zhou S, Schuetz J D, Bunting K D, et al. The ABC transporter Bcrp1/ABCG2 is expressed in a wide variety of stem cells and is a molecular determinant of the side-population phenotype. Nat Med. 2001;7:1028-1034.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

TABLE 1
Multiparameter cytometry for measurement of outcomes (differentiation markers, MDR, apoptosis,
self-renewal) on analytic classifier populations (stem, progenitor, mature) and sorted subsets.
Outcomes will be measured in two contexts: 1) Further characterization of sorted populations (flow
or ArrayScan V^{TI}); 2) Characterization of sorted cells after culture (ArrrayScan V^{TI}).

| | Laser | | | | | | | | | | |
|---|---|---|---|---|---|---|---|---|---|---|---|
| | | | | | | 635 Innova | | 351 Enterprise | | 405 Violet | |
| | 488 Enterprise/Diode | | | | | Spectrum/Diode | | (MoFlo only) | | (CyAn only) | |
| PMT | FL₁ | FL₂ | FL₃ | FL₄ | FL₅ | FL₆ | FL₇ | FL₈ | FL₉ | FL₈ | FL₉ |
| Fluorochromes | R123 | PE | ECD | PC5 | PC7 | APC | APC7 | Ho Blue | Ho Red | CY | PB |
| Antigens | MDR1 | CD45 or | CD90 | ABCG2 | CD117 | HEA | CD133 | SP | SP | Outcome | Outcome |
| | | lineage + PI | | | | | | | | | |
| FL8 and FL9 are used for the uv laser on the MoFlo sorter, and for the violet laser on the CyAn analyzer. Which instrument we use, and how many outcomes we can measure simultaneously is instrument dependent. Antigens used for the identification drug resistance are shown in bold. The remaining markers are used to distinguish stem, progenitor and mature cells. | | | | | | | | | | | |
| # Additional parameters will be read as outcomes. PE = phycoerythrin, ECD = PE-Texas red, PC5 = PE-Cyanine5, PC7 = PE-Cyanine7, APC = allophycocyanin, APC7 = AP-Cyanine7, Ho = Hoechst33342, CY = Cascade Yellow, PB = Pacific Blue, PI = propidium idodide | | | | | | | | | | | |

TABLE 2
Outcome markers of stem cell and neuroectodermal and
epithelial differentiation to be measured on classifier
populations (stem, progenitor, mature).

| Stage | Antigen | Cells that Normally Express Marker |
|---|---|---|
| Embryonic | Oct-4 | Pluripotent stem |
| | SSEA3 | Pluripotent stem |
| Neurectoderm | p73 | Neuroectoderm and neural stem |
| | nestin | Neuroectoderm and neural stem |
| | notch1 | Neural progenitor |
| Epith. SC | KRT15 | Cross-reactive to keratin 15 |
| | p63 | Fetal and adult epithelial stem |
| | CD200 | Epithelial stem |
| Epith. PC | CD24 | Non-bulge hair follicle cells |
| | CD71 | Proliferating epithelial progenitors |
| | EDNR | Endothelin receptor |
| Mature | HEA | Basolateral surface of epithelial cells |
| | MUC-1 | Luminal surface of secretory cells |
| | Keratins | Mature epithelium |

1. An isolated multiple drug resistance (MDR) cancer stem cell substantially isolated from non-MDR cancer stem cells of the same species of the MDR cancer cell.

2. The isolated MDR cancer stem cell of claim 1, which is tumorigenic at high frequency in a xenograft model.

3. The isolated MDR cancer stem cell of claim 1, which is human.

4. The isolated MDR cancer stem cell of claim 3, which is tumorogenic at high frequency in a mouse xenograft model.

5. The isolated MDR cancer stem cell of claim 3, which is tumorigenic at a frequency of a least about 40 cells per injection site in SCID/NOD mice.

6. The isolated MDR cancer stem cell of claim 1, which bears stem-cell associated markers.

7. The isolated MDR cancer stem cell of claim 1, which bears progenitor-cell associated markers.

8. The isolated MDR cancer stem cell of claim 1, which is constitutively protected by MDR transporters.

9. The isolated MDR cancer stem cell of claim 1, which is CD45−.

10. The isolated MDR cancer stem cell of claim 1, which is CD90+.

11. The isolated MDR cancer stem cell of claim 1, which is CD117+.

12. The isolated MDR cancer stem cell of claim 1, which is CD133+.

13. The isolated MDR cancer stem cell of claim 1, which expresses ABCG2.

14. The isolated MDR cancer stem cell of claim 1, which excludes rhodamine 123.

15. The isolated MDR cancer stem cell of claim 1, which excludes Hoechst 33342.

16. A population comprising one or more MDR cancer stem cell(s) of claim 1.

17. The population of clam 16, which is substantially homogenous.

18. The population of claim 16, which is clonal or clonogenic.

19. The population of claim 16, which is clonogenic in vitro.

20. The population of claim 16, which is in vivo within an animal other than the species of the MDR cancer stem cell population.

21. A method of identifying an MDR cancer stem cell, the method comprising:
a. obtaining a tissue sample from a patient,
b. staining single cells from the biopsy with dye-conjugated antibodies for identification or purification by flow cytometry, wherein the antibodies target one or more hematopoetic stem/progenitor markers, CD90, CD117, CD133, and a marker of multiple drug resistance,
c. optionally culturing the stained cells in the presence of one or more fluorescent MDR substrates,
d. optionally adding a viability dye to the cells,
e. subjecting the cells to flow cytometry within a short period of time following the addition of the viability dye;
whereby the MDR cancer stem cell is identified as having a plurality of the following factors: 1) Live (viability dye excluding); 2) Singlet (by forward light scatter pulse analysis; 3) Non-hematopoietic; +; 5) CD90, CD117 and/or CD133 positive; 6) MDR expression and/or activity by appositive staining for the marker of multiple drug resistance and/or transport of a fluorescent MDR substrate.

22. The method of claim 21, wherein the marker of multiple drug resistance is ABCG2, ABCB1, ABCC1, or Lung Resistance Protein (LRP).

23. The method of claim 21, wherein a hemopoetic marker is CD45.

24. The method of claim 21, wherein the fluorescent MDR substrate is Rhodamine 123 and/or Hoechst 33342.

25. A method of isolating an MDR cancer stem cell, the method comprising identifying an MDR cancer stem cell in accordance with the method of claim 21, and further placing the cell in a suitable culture medium to maintain viability.

26. A method of screening a test compound, the method comprising
a. culturing a test population of MDR cancer stem cell(s) of any of claims 16-18,
b. exposing the test population to a test compound, and
c. assaying for the effect of the test compound on the viability or proliferation of the test population, or for the change in phenotypic profile of the test population away from the MDR cancer stem cell phenotype;
whereby the ability of the test compound to kill cells within the test population, to retard the proliferation of the test population, or sensitize the cells to other compounds or to change the phenotypic profile of the test population away from the MDR cancer stem cell phenotype, identifies the test compound as a potential anti-cancer agent effective against MDR cancer stem cells.

27. The method of claim 26, wherein a plurality of test populations is employed.

28. The method of claim 26, wherein a plurality of test compounds is employed.

29. The method of claim 28, wherein each separate test population of the plurality of populations is cultured in a well of a multi-well plate.

30. The method of claim 29, where separate test populations among the plurality of populations is/are exposed to a distinct test compound.

31. The method of claim 30, where separate test population of the plurality of populations is/are exposed to a different concentrations of the same test compound.

32. A method of assaying for the presence of a target molecule on the surface of a cancer stem cell comprising exposing the population of cancer stem cells of any of claims 16-18 to a ligand recognizing the target molecule under conditions for the ligand to specifically bind the target molecule to form and thereafter assaying for ligand-binding events.

33. The method of claim 32, wherein the ligand is an antibody or portion thereof.

34. The method of claim 32, wherein the ligand is a hormone or portion thereof.

35. The method of claim 32, wherein the target molecule is a growth factor receptor or an antigenic determinant.
